(19) United States
(12) Patent Application Publication
(10) Pub. No.: US 2025/0161475 A1
(43) Pub. Date: May 22, 2025

(54) TNFALPHA IMMUNOCONJUGATE PREPARATION

(71) Applicant: PHILOGEN S.P.A, Siena (IT)

(72) Inventors: Ettore GILARDONI, Otelfingen (CH); Paola BATTESTIN, Siena (IT); Camilla BACCI, Siena (IT); Alessandro REDI, Siena (IT); Emiliano SANI, Siena (IT); Riccardo STUCCHI, Otelfingen (CH); Roberto DE LUCA, Otelfingen (CH); Cesare DI NITTO, Otelfingen (CH); Mattia MATASCI, Otelfingen (CH); Dario NERI, Siena (IT)

(21) Appl. No.: 18/841,662

(22) PCT Filed: Mar. 21, 2023
(86) PCT No.: PCT/EP2023/057241

§ 371 Date: Aug. 26, 2024

(30) Foreign Application Priority Data
Mar. 21, 2022 (EP) 22163383.7

(51) Int. Cl. A61K 47/68; C07K 16/18

(52) U.S. Cl. CPC A61K 47/6813; A61K 47/6843; C07K 16/18

(57) ABSTRACT

The application relates to a preparation, in particular a pharmaceutical preparation, comprising a non-glycosylated TNFα immunoconjugate and a glycosylated TNFα immunoconjugate, wherein the percentage of the glycosylated TNFα immunoconjugate of the total TNFα immunoconjugate in the preparation is low, preferably 10% or lower. The preparation finds application in the treatment of cancer, such as brain tumors, in human patients.

This application claims priority from European Patent Application No. 22163383.7 filed on 21 Mar. 2022, the contents and elements of which are herein incorporated by reference for all purposes.

### FIELD OF THE INVENTION

The present invention relates to a preparation, in particular a pharmaceutical preparation, comprising a non-glycosylated TNFα immunoconjugate and a glycosylated TNFα immunoconjugate, wherein the percentage of the glycosylated TNFα immunoconjugate of the total TNFα immunoconjugate in the preparation is low, preferably 10% or lower. The preparation finds application in the treatment of cancer, such as brain tumors, in human patients.

### BACKGROUND

Current applicants described in WO2001/062298, which is hereby incorporated by reference in its entirety, an immunoconjugate comprising human TNFα fused to antibody L19 (“L19-TNFα”). L19 (U.S. Pat. No. 8,097,254) specifically binds the ED-B domain of fibronectin isoform B-FN, which is one of the best-known markers of angiogenesis. ED-B is an extra domain of 91 amino acids found in the B-FN isoform. ED-B accumulates around neovascular structures in aggressive tumours and other tissues undergoing angiogenesis, such as the endometrium in the proliferative phase and some ocular structures in pathological conditions but is otherwise undetectable in normal adult tissues. A number of uses, including the treatment of human cancers, and formulations of L19-TNFα have been previously described by current applicants in WO2013/045125, WO2018/011404, WO2019/185792, WO2021/234178, which are hereby incorporated by reference in their entirety.

Large-scale production of immunoconjugates, such as L19-TNFα can be achieved by recombinant expression. However, prokaryotic (bacterial) expression is unsuitable for the expression of complex proteins such as L19-TNFα which forms a large 150 kDa non-covalent homotrimer in solution.

Borsi et al. Blood (2003) 102, 4384 detail the use SP2/0 murine myeloma cells to express recombinant L19-murine (m) TNFα, reporting a final yield of 3 mg/L of purified protein which is not suitable for industrial production of the conjugate.

In light of the above, there is a need in the art for additional methods for large-scale production of L19-human TNFα suitable for the treatment of human patients.

### SUMMARY OF THE INVENTION

The present inventors have devised a production method for the preparation of L19-TNFα, which has a high yield and, surprisingly, results in a low percentage of glycosylated L19-TNFα in the preparation. Specifically, the percentage of glycosylated L19-TNFα in the preparation was reported to be between 3% and 5.36% (FIG. 5A), with an average of 4.19% of the L19-TNFα being glycosylated (FIG. 5B).

Furthermore, preparations comprising a low percentage of glycosylated L19-TNFα are expected to have superior biological activity in comparison with preparations comprising a high percentage of glycosylated L19-TNFα. Specifically, L19-TNFα conjugates which are not glycosylated are expected to have increased affinity for Tumor Necrosis Factor Receptor 1 (TNFR1) and or Tumor Necrosis Factor Receptor 2 (TNFR2) relative to glycosylated L19-TNFα conjugates.

Preparations comprising a low percentage of glycosylated L19-TNFα are also expected to have superior thermostability and/or storage stability in comparison with preparations comprising a high percentage of glycosylated L19-TNFα. Specifically, L19-TNFα conjugates which are not glycosylated are expected to have increased thermostability relative to glycosylated L19-TNFα conjugates.

Still further, preparations comprising a low percentage of glycosylated L19-TNFα are expected to be less susceptible to protease-mediated degradation in comparison with preparations comprising a high percentage of glycosylated L19-TNFα. Specifically, L19-TNFα conjugates which are not glycosylated are expected to be less susceptible to protease-mediated degradation relative to glycosylated L19-TNFα conjugates.

Preparations comprising a low percentage of glycosylated L19-TNFα are also expected to be more suitable for lyophilisation in comparison with preparations comprising a high percentage of glycosylated L19-TNFα.

Without wishing to be bound by theory, it is believed that the fermentation conditions used to prepare the L19-TNFα conjugate described herein result in the low level of glycosylated species observed.

High levels of glycosylation present challenges in terms of batch-to-batch reproducibility for good manufacturing practice (GMP) production, increased immunogenicity and suboptimal pharmacokinetics when preparing a product for industrial development. Durocher & Butler, Current Opinion Biotechnol (2009), 20, 770 explain that glycan structures may be immunogenic and that immunogenicity can reduce the efficacy of a biologic through rapid clearance by immune system. Planinc et al. Analytica Chimica Acta (2016) 13, 27 similar confirm the importance of low abundance of glycans due to their potential immunogenicity. Leepenies & Seeberger Nature Biotechnol. (2016), 32, 443 further comment that heterogeneous glycosylation can result in batch-to-batch variations in efficacy or pharmacokinetics.

In view of the drawbacks associated with glycosylation of immunoconjugates such as L19-TNFα detailed above, the low percentage of glycosylated L19-TNFα in the preparation resulting from the production method devised by the present inventors is expected to have advantageous properties when used in the treatment of human patients compared with L19-TNFα preparations comprising higher percentages of glycosylated L19-TNFα.

The low level of glycosylated L19-TNFα in the preparation was surprising in view of the fact that production of human TNFα when prepared in the same cell line (Chinese Hamster Ovary) had previously been shown to result in high levels of glycosylation. Specifically, in WO98/55662, expression of recombinant non-conjugated human TNFα in Chinese Hamster Ovary (CHO) cells resulted in a TNFα preparation in which 35% of the total TNFα was reported to be O-glycosylated. The authors consider this to be advantageous on the basis that glycosylated

TNFα may have an increased half-life in body fluids, may improve binding to receptors and may be better protected against the influence of proteases than non-glycosylated TNFα. In Takakura et al., Eur J Bioch (1996), 235, 431, the human B-cell lymphoblastoid cell line BALL-1 was used to express recombinant human TNFα. Consistent with the high level of glycosylation reported in WO98/55662, the percentage of O-glycosylated TNFα in the resulting preparation was reported to be 20%. The O-glycosylation was further reported to be linked to the serine at position 4 of the amino acid sequence of human TNFα.

Thus, in a first aspect, the present invention provides a preparation comprising:
(i) a non-glycosylated TNFα immunoconjugate; and
(ii) a glycosylated TNFα immunoconjugate;
wherein the TNFα immunoconjugate comprises human TNFα conjugated to an antibody molecule comprising the L19 complementarity determining regions (CDRs) set forth in SEQ ID NOs 1 to 6, and
wherein the percentage of the glycosylated TNFα immunoconjugate is ≤10% of the total TNFα immunoconjugate in the preparation.

In a preferred embodiment, the antibody molecule in the TNFα immunoconjugate comprises the L19 VH domain set forth in SEQ ID NO: 7 and the L19 VL domain set forth in SEQ ID NO: 9. More preferably, the antibody molecule has the sequence of the L19 antibody in scFv format set forth in SEQ ID NO: 10. Most preferably, the TNFα immunoconjugate consists of or comprises the sequence of L19-TNFα set forth in SEQ ID NO: 13.

The TNFα is preferably human TNFα. Most preferably, the TNFα has the sequence set forth in SEQ ID NO: 11.

Thus, in a preferred embodiment, the present invention provides a preparation comprising:
(i) non-glycosylated L19-TNFα; and
(ii) glycosylated L19-TNFα;
wherein the L19-TNFα comprises or consists of the sequence set forth in SEQ ID NO: 13; and
wherein the percentage of glycosylated L19-TNFα is ≤10% of the total L19-TNFα in the preparation.

The percentage of glycosylated TNFα immunoconjugate of the total TNFα immunoconjugate in the preparation is preferably ≤10%, ≤9%, ≤8%, ≤7%, or ≤6%, with a lower % of glycosylation being preferred for the reasons explained above.

The percentage of glycosylated TNFα immunoconjugate of the total TNFα immunoconjugate in the preparation may be ≥2%, or ≥3%.

For example, the percentage of glycosylated TNFα immunoconjugate of the total TNFα immunoconjugate in the preparation may be ≥3% and ≤6%.

The glycosylated TNFα immunoconjugate preferably comprises an O-linked glycosylation at the serine at position 4 of TNFα, wherein the TNFα has the sequence set forth in SEQ ID NO: 11. Where the TNFα immunoconjugate is L19-TNFα and comprises or consists of the sequence set forth in SEQ ID NO: 13, the glycosylated L19-TNFα preferably comprises an O-linked glycosylation on the serine at position 257 of SEQ ID NO: 13. The O-linked glycosylation preferably comprises one core of hexose-acetyl-hexose 1 and one N-acetylneuraminic acid residue. Most preferably, the O-linked glycosylation is HexNAc₁Hex₁NeuAc₁. In a preferred embodiment, the TNFα immunoconjugate is not glycosylated at any other position in the TNFα sequence.

In a preferred embodiment, the present invention provides a preparation comprising:
(i) non-glycosylated L19-TNFα; and
(ii) glycosylated L19-TNFα;
   wherein the L19-TNFα comprises or consists of the sequence set forth in SEQ ID NO: 13, wherein the percentage of glycosylated L19-TNFα is ≤6% of the total L19-TNFα in the preparation, and
   wherein the preparation has a higher biological activity compared to a preparation in which the percentage of glycosylated L19-TNFα is 15% or more of the total L19-TNFα in the preparation.

In a preferred embodiment, the preparation has increased affinity for Tumor Necrosis Factor Receptor 1 (TNFR1) and or Tumor Necrosis Factor Receptor 2 (TNFR2) relative to a preparation in which the percentage of glycosylated L19-TNFα is 15% or more of the total L19-TNFα in the preparation.

In a preferred embodiment, the preparation has superior thermostability and/or storage stability compared to a preparation in which the percentage of glycosylated L19-TNFα is 15% or more of the total L19-TNFα in the preparation.

In a preferred embodiment, the preparation is less susceptible to protease-mediated degradation compared to a preparation in which the percentage of glycosylated L19-TNFα is 15% or more of the total L19-TNFα in the preparation.

In a preferred embodiment, the preparation is more suitable for lyophilisation compared to a preparation in which the percentage of glycosylated L19-TNFα is 15% or more of the total L19-TNFα in the preparation.

Methods for determining the type and percentage of glycosylated TNFα immunoconjugate in a preparation of the TNFα immunoconjugate are known to the skilled person and include mass spectrometry, in particular intact mass analysis, as described herein.

The pharmaceutical preparation of the invention may further comprise, in addition to the active ingredient (glycosylated and non-glycosylated TNFα immunoconjugate), a pharmaceutically acceptable excipient, carrier, buffer, stabiliser or other materials well known to those skilled in the art and suitable for the administration to human patients. Such materials should be non-toxic and should not interfere with the efficacy of the active ingredient. For injection at a tumour site, the pharmaceutical preparation may be in the form of a parenterally acceptable aqueous solution which is pyrogen-free and has suitable pH, isotonicity and stability.

The pharmaceutical preparation of the invention may be for use in a method of treating cancer in a patient. Also provided is a method of treating cancer, the method comprising administering a therapeutically effective amount of the pharmaceutical preparation of the invention to the patient. Similarly provided is the use of the pharmaceutical preparation of the invention in the manufacture of a medicament for the treatment of cancer. The patient is preferably a human patient.

Cancers that may be treated using a pharmaceutical preparation of the invention include skin cancer, such as a malignant melanoma or non-melanoma skin cancer, sarcoma, such as soft-tissue sarcoma, and brain tumors such as glioma or glioblastoma.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows the general outline of the production process of L19-TNFα used to obtain the pharmaceutical preparation comprising a low percentage of glycosylated L19-TNFα of the invention.

FIG. 2 shows the general fermentation procedure of the production process for L19-TNFα used to obtain the pharmaceutical preparation comprising a low percentage of glycosylated L19-TNFα of the invention.

FIGS. 3A and B: bidimensional SDS-PAGE analysis of the pharmaceutical preparation comprising a low percentage of glycosylated L19-TNFα confirmed the purity of the sample after production, as well as the molecular weight (˜45 KDa) and isoelectric point (˜7.7) of L19-TNFα. Two spots were visible: non-glycosylated L19-TNFα (spot 1) and glycosylated L19-TNFα (spot 2). Non-glycosylated L19-TNFα was the most intense spot, while the spot corresponding to glycosylated L19-TNFα was barely visible, indicating a very low abundance of the glycosylated species. C: purified milligrams of L19-TNFα per litre of fermentation culture recovered from seven lots of production.

FIG. 4 shows the deconvoluted spectrum of the mass spectrometry analysis of a L19-TNFα preparation comprising a low percentage of glycosylated L19-TNFα prepared according to the method described herein. The main peak (1) at 43957 Da corresponds to the non-glycosylated L19-TNFα conjugate while peak (2) at 44613 Da corresponds to the L19-TNFα variant with an O-linked glycosylation at position 257. The other minor peaks are irrelevant: the small peak at 43913 Da is a typical MS artefact corresponding to L19-TNFα without one CO₂ group. The other small peak at 44056 Da corresponds to L19-TNFα in aggregation with a phosphate group of the buffer.

FIG. 5A shows the percentage of non-glycosylated L19-TNFα and glycosylated L19-TNFα of the total L19-TNFα in different production lots of the pharmaceutical preparation prepared using the method described herein. In seven lots of production using the method described herein, glycosylated L19-TNFα never exceeded 6% of the total L19-TNFα in the preparation, with an average percentage of glycosylated L19-TNFα in the preparation of 4.19% (FIG. 5B).

FIG. 6 shows the MS1 spectrum of the glycosylated TNFα peptide SSSRTPSDKPVAHVVANPQAEGQLQWLNR in a representative lot of production of an L19-TNFα preparation comprising a low percentage of glycosylated L19-TNFα. Detection of a 4+charged ion at 958.21 m/z is consistent with the presence of one additional NeuAc group on the HexNAcHex1 core attached to the serine at position 2 of the TNFα peptide which corresponds to the serine at position 257 in the L19-TNFα conjugate.

FIG. 7 shows the MS2 spectrum of the glycosylated TNFα peptide SSSRTPSDKPVAHVVANPQAEGQLQWLNR in a representative lot of production of an L19-TNFα preparation comprising a low percentage of glycosylated L19-TNFα. The precursor ion at 958.21 m/z has been isolated and HCD fragmented to generate this MS2 spectrum. Together with the expected list of y-ions, the presence of two peaks at 274.09 m/z (NeuAc-H₂O) and 292.10 m/z (NeuAc) confirms the presence of the NeuAc group on the peptide. This data further confirms the correct identification of the HexNAc₁Hex₁NeuAc₁ group on the serine at position 2 of the peptide, which corresponds to the serine at position 257 of L19-TNFα conjugate.

### DETAILED DESCRIPTION

Aspects and embodiments of the present invention will now be discussed with reference to the accompanying figures. Further aspects and embodiments will be apparent to those skilled in the art. All documents mentioned in this text are incorporated herein by reference.

The pharmaceutical composition of the present invention comprises a TNFα immunoconjugate, wherein the TNFα immunoconjugate comprises an antibody molecule comprising the L19 complementarity determining regions (CDRs) set forth in SEQ ID NOs 1 to 6 and TNFα.

The antibody molecule is preferably monoclonal. The antibody molecule may be human or humanised, but preferably is a human antibody molecule.

The TNFα immunoconjugate may be isolated, in the sense of being free from contaminants, such as antibodies able to bind other polypeptides, and/or serum components.

The antibody molecule may be natural or partly or wholly synthetically produced. For example, the antibody molecule may be a recombinant antibody molecule.

The antibody molecule may be an immunoglobulin, or an antigen-binding fragment thereof. For example, the antibody molecule may be an IgG, IgA, IgE or IgM molecule, preferably an IgG molecule, such as an IgG1, IgG2, IgG3 or IgG4 molecule, but more preferably is an antigen-binding fragment thereof. In a more preferred embodiment, the antibody molecule comprises or consists of a single-chain Fv (scFv), a small immunoprotein, a diabody, but most preferably is an scFv.

The antibody molecule preferably comprises the L19 VH domain set forth in SEQ ID NO: 7 and/or the L19 VL domain set forth in SEQ ID NO: 9. More preferably, the antibody molecule has the sequence of the L19 antibody in scFv format set forth in SEQ ID NO: 10.

Where the antibody molecule is an scFv, the VH and VL domains of the antibody are preferably linked by a 12 to 20 amino acid linker. For example, the VH and VL domains may be linked by an amino acid linker which is 12, 13, 14, 15, 16, 17, 18, 19, or 20 amino acid in length. Suitable linker sequences are known in the art and include the linker sequence set forth in SEQ ID NO: 8.

The TNFα is preferably human TNFα. Most preferably, the TNFα comprises or consists of the sequence set forth in SEQ ID NO: 11.

The antibody molecule, e.g. in scFv format, and the TNFα may be connected to each other directly, for example through any suitable chemical bond, but preferably are connected via a peptide linker. The chemical bond may be, for example, a covalent or ionic bond. Examples of covalent bonds include peptide bonds (amide bonds) and disulphide bonds.

Where the TNFα is connected to the antibody molecule via a peptide linker, the peptide linker may be a short (2-30, preferably 10-20) residue stretch of amino acids. Suitable examples of peptide linker sequences are known in the art. One or more different linkers may be used. An exemplary linker sequence is set forth in SEQ ID NO 12. In one embodiment, the linker may be a cleavable linker.

Where the antibody molecule and TNFα are connected via a peptide bond or peptide linker, the conjugate may be produced (secreted) as a single chain polypeptide, such as a fusion protein.

In a preferred embodiment, the TNFα is conjugated to the C-terminus of the antibody molecule in scFv format. Most preferably, the TNFα immunoconjugate consists of, or comprises, the sequence of L19-TNFα set forth in SEQ ID NO: 13.

The TNFα in the glycosylated TNFα immunoconjugate comprised in the pharmaceutical preparation of the invention, preferably comprises an O-linked glycosylation on the serine at position 4 of the TNFα sequence set forth in SEQ ID NO: 11. Where the TNFα immunoconjugate is L19-TNFα and comprises or consists of the sequence set forth in SEQ ID NO: 13, the glycosylated L19-TNFα preferably comprises an O-linked glycosylation on the serine at position 257 of SEQ ID NO: 13.

The O-linked glycosylation comprises one core of hexose-acetyl-hexose 1 and one N-acetylneuraminic acid residue. Preferably, the O-linked glycosylation is HexNAc₁Hex₁NeuAc₁. In a preferred embodiment, the TNFα immunoconjugate is not glycosylated at any other position in the TNFα sequence.

The L19 in the glycosylated L19-TNFα immunoconjugate comprised in the pharmaceutical preparation of the invention is not glycosylated. That is, only the TNFα in the glycosylated L19-TNFα immunoconjugate comprised in the pharmaceutical preparation of the invention is glycosylated.

The percentage of glycosylated TNFα immunoconjugate of the total TNFα immunoconjugate in the pharmaceutical preparation is preferably ≤10%, ≤9.5%, ≤9%, ≤8.5%, ≤8%, ≤7.5%, ≤7%, ≤6.5%, ≤6%, or ≤5.5%, with a lower % of glycosylation being preferred for the reasons explained above. In a preferred embodiment, the % of glycosylation is ≤6%, more preferably ≤ 5.5%.

The percentage of glycosylated TNFα immunoconjugate of the total TNFα immunoconjugate in the preparation may be ≥2%, ≥2.5%, or ≥3%. This excludes TNFα immunoconjugate made in bacterial cells, which are not glycosylated.

For example, the percentage of glycosylated TNFα immunoconjugate of the total TNFα immunoconjugate in the preparation may be ≥2% and ≤10%, ≥2% and ≤6%, ≥3% and ≤6%, ≥2% and ≤5.5%, ≥3% and ≤5.5%.

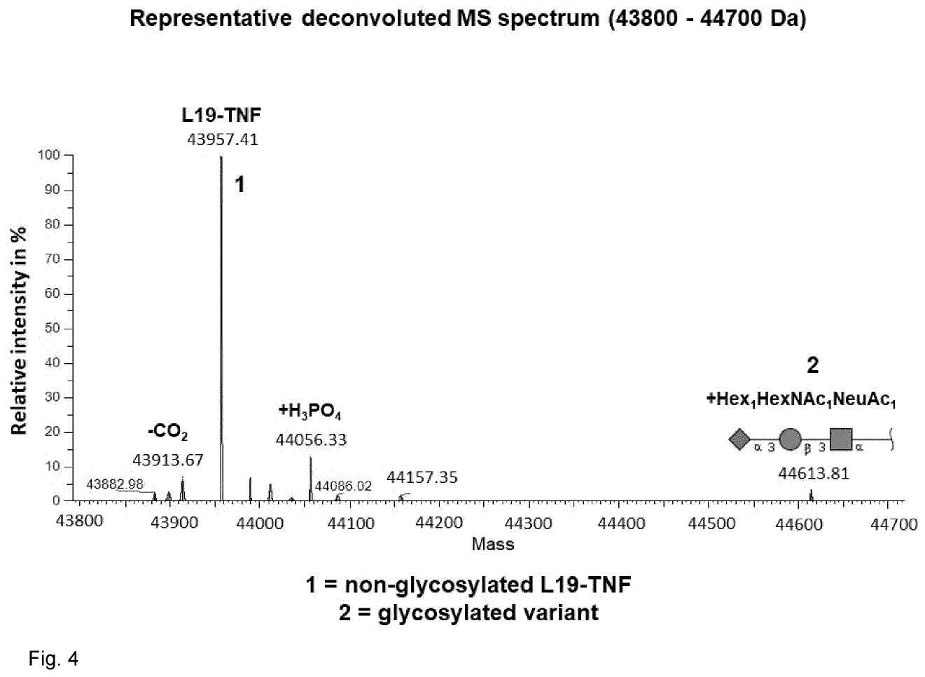

Methods for measuring the percentage of glycosylated TNFα immunoconjugate present in a pharmaceutical composition, as well as determining the type of glycosylation present are known in the art and include mass spectrometry, in particular intact mass analysis. An exemplary method is detailed in Example 3. The results of said analysis are shown in FIGS. 4 to 7.

The pharmaceutical preparation of the invention may comprise, in addition to glycosylated and non-glycosylated TNFα immunoconjugate, as set out in the claims, a pharmaceutically acceptable excipient, carrier, buffer, stabiliser or other materials well known to those skilled in the art and suitable for the administration to human patients. The pharmaceutical composition may comprise a buffer composition as disclosed in WO2018/011404. In particular, the pharmaceutical composition may comprise glycosylated and non-glycosylated TNFα immunoconjugate, as set out in the claims, dissolved in a sodium phosphate buffer which comprises a salt at a concentration of at least about 1.5 mM, polysorbate at a concentration of at least about 0.005% (v/v), and a stabilizer, wherein the pH of the sodium phosphate buffer is higher than 7.5 and lower than 9. Preferably, the polysorbate concentration is about 0.1% (v/v) or less. The sodium phosphate buffer preferably comprises glycerol at about 0.5-1.5% w/v. The sodium phosphate buffer may further comprises NaH2PO4 at a concentration of about 5-25 mM. The sodium phosphate buffer may further comprises Na2HPO4 at a concentration of about 5-20 mM. The sodium phosphate buffer may also comprise KCl at a concentration of about 1-2 mM. Additionally, the sodium phosphate buffer may comprise EDTA at a concentration of about 1-20 mM. The stabilizer may be a sugar, preferably mannitol, trehalose, sucrose, sorbitol, maltose or xylitol, most preferably mannitol. The sugar may be present at a concentration of 20-250 mM. The salt may be NaCl and may be present in the sodium phosphate buffer at a concentration of about 10-30 mM. The sodium phosphate buffer preferably comprises polysorbate at a concentration of about 0.005 to about 0.03%. The polysorbate may be polysorbate20. In a preferred embodiment, the concentration of the total TNFα immunoconjugate (glycosylated and non-glycosylated) in the pharmaceutical preparation may be is at least about 0.2 mg/mL, more preferably at least about 0.4 mg/mL. In a most preferred embodiment, the pharmaceutical preparation of the invention comprises, in addition to glycosylated and non-glycosylated TNFα immunoconjugate, NaH₂PO₄(2H₂O) at a concentration of 15 mM, Na₂HPO₄ (2H₂O) at a concentration of 10 mM, KCl at a concentration of 1.5 mM, mannitol at a concentration of 75 mM, NaCl at a concentration of 30 mM, 1% w/v glycerol, EDTA at a concentration of 5 mM, 0.01% v/v Tween20, and has a pH of 8.00.

The preparation of the invention is expected to have a higher biological activity compared to a preparation in which the percentage of glycosylated L19-TNFα is 15% or more, 20% or more, 25% or more, or 30% or more, of the total L19-TNFα in the preparation.

In one embodiment, the preparation is expected to have a higher affinity for TNFR1 and/or TNFR2 as measured by BiaCore or other suitable techniques.

In some embodiments, the pharmaceutical preparation of the invention may be administered alone or in combination with other treatments, either simultaneously or sequentially dependent upon the condition to be treated.

Treatments involving the pharmaceutical preparation of the invention may include the administration of a second anti-cancer therapy. Many suitable anti-cancer therapies are known in the art. For example, L19-TNFα has been successfully used in anti-cancer therapy when administered in combination with doxorubicin, dacarbazine, radiotherapy and temozolomide, or lomustine (WO2021/234178). Clinical trials for these combinations are ongoing. Promising anti-cancer effects have also been reported for the combined administration of L19-TNFα and L19-IL2 (WO2018/011404). This is in addition to the anti-cancer effect demonstrated for L19-TNFα monotherapy.

Thus, the pharmaceutical preparation of the invention may be for use in a method of treating cancer in a patient, wherein the method further comprises administering a second anti-cancer therapeutic selected from the group consisting of doxorubicin, dacarbazine, radiotherapy and temozolomide, lomustine, or L19-IL2, to the patient. Administration of the pharmaceutical preparation of the invention and the second anti-cancer therapy to the patient may be simultaneous or sequential, whereby simultaneous administration refers to administration in the same treatment cycle but not necessarily on the same day.

The pharmaceutical preparation of the invention and the second anti-cancer therapy may be provided as a combined preparation but are preferably provided as separate preparations to permit either simultaneous or sequential administration. Where the treatment further involves radiotherapy, this will necessarily be administered separately from the pharmaceutical preparation of the invention but administration may nonetheless be simultaneous.

Further treatments may be used in combination with the pharmaceutical preparation of the invention include the administration of suitable doses of pain relief drugs such as non-steroidal anti-inflammatory drugs (e.g. aspirin, paracetamol, ibuprofen or ketoprofen) or opiates such as morphine, or antiemetics.

Where the pharmaceutical preparation of the invention is administered for cancer treatment, it may be injected parenterally. In one embodiment, the pharmaceutical preparation of the invention is injected at the site of the tumour, preferably by intratumoral injection. Peritumoral injection, e.g. local intradermal injection, is another suitable method for administering the pharmaceutical preparation of the invention locally to a tumour site. In some embodiments, the pharmaceutical preparation of the invention may be administered by infusion, e.g. intravenous infusion.

Cancer treatment according to the present invention may include complete eradication of the tumour. The disappearance of any evidence of vital tumour after termination of the treatment represents complete treatment of the tumour. Disappearance of the tumour may be determined when the tumour has no discernible volume or is no longer visible. Treatment may comprise treatment to eradicate the tumour and prevent tumour regrowth.

Patients are preferably monitored during a follow-up period of at least one month, preferably at least six months or at least a year, after administration of the pharmaceutical preparation of the invention. Disappearance of the tumour, and lack of tumour regrowth, may be observed in the follow-up period. Absence of tumour regrowth may be observed.

The quantity of L19-TNFα administered through administration of the pharmaceutical preparation of the invention to the patient will depend on the size and nature of the tumour, among other factors. The determination of suitable doses is within the competence of the skilled practitioner. Suitable doses are disclosed in WO2021/234178, WO2018/011404, and WO2013/045125. In particular, L19-TNFα can be given at doses between 5 and 20 μg/Kg, preferably between 6 and 18 μg/Kg, between 7 and 17 μg/Kg or between 8 and 15 μg/Kg, more preferably between 10 and 13 μg/Kg. Preferably, the dose of L19-TNFα is in the range of 200 μg to 400 μg. Most preferably, the dose of L19-TNFα is 400 μg. These are examples only and different doses may be used.

The pharmaceutical preparation of the invention may be for use in a method of treating cancer. The cancer may be a solid tumor. In a preferred embodiment, the cancer is selected from a skin cancer such as malignant melanoma, non-melanoma skin cancer such as Basal Cell Carcinoma (BCC), Squamous Cell Carcinoma (cSCC), glioma, such as a glioblastoma, or sarcoma, such as soft-tissue sarcoma. The cancer may be metastatic or non-metastatic. The cancer may be recurrent or non-recurrent, such as recurrent glioblastoma.

Where the cancer to be treated with the pharmaceutical preparation of the invention is a glioma, such as a glioblastoma, the pharmaceutical preparation is preferably administered together with lomustine and, optionally, a further chemotherapy. Details of such a therapy are disclosed in WO2021/234178, which is hereby incorporated by reference in its entirety. Thus, the pharmaceutical preparation of the invention may be for use in a method of treating a glioma, such a glioblastoma, in a patient, wherein the method further comprises administering lomustine and optionally, a second chemotherapy, such as temozolomide, to the patient.

Where the cancer to be treated with the pharmaceutical preparation of the invention is a skin cancer, the pharmaceutical preparation is preferably administered together with L19-IL2. The L19-IL2 preferably comprises or consists of the sequence set forth in SEQ ID NO: 14. Details of such a therapy are disclosed in WO2013/045125, which is hereby incorporated by reference in its entirety. Thus, the pharmaceutical preparation of the invention may be for use in a method of treating skin cancer, such as a malignant melanoma or non-melanoma skin cancer, but preferably a malignant melanoma, in a patient, wherein the method further comprises administering L19-IL2 to the patient. Administration may be by injection at the tumor site, in particular, by intra-tumoral injection. Most preferably, the dose of L19-IL2 is 2.17 mg. The dose of L19-IL2 can alternatively be stated in international units (IU). 2.17 mg L19-IL2 equates to 13 million IU L19-IL2. In the case of intra-tumoral injections, patients may be treated with 13 million IU of L19-IL2 corresponding to 2.17 mg of L19-IL2 and with 400 μg of L19-TNFα once weekly for one to four weeks, e.g., for one, two, three or four consecutive weeks. The dose may be administered as a single intra-tumoral injection, or a dose may be divided into multiple intratumoral injections, which are administered to the same tumor. Alternatively, in certain cases the dose may halved to be 1.08 mg of L19-IL2 and 200 μg of L19-TNFα.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps. It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—Method for Producing a Preparation of L19-TNFα Comprising a Low Percentage Glycosylated L19-TNFα

### 1.1 General Description of Manufacturing Process and Process Controls

L19-TNFα is a recombinant fusion protein stably expressed in in Chinese Hamster Ovary host cells (CHO-K1), cultured in bioreactors using a semi-continuous fermentation approach. L19-TNFα is recovered from the harvested medium by several chromatographic purification steps.

The manufacturing process of L19-TNFα Drug Substance can be divided into two upstream phases and two downstream phases.
Upstream 1 (phase A): inoculum preparation
Upstream 2 (phase B): fermentation
Downstream 1 (phase C): capture purification by affinity chromatography
Downstream 2 (phase D): polishing and formulation by gel filtration, cation exchange chromatography, buffer exchange, anion exchange chromatography, nanofiltration and a final concentration.

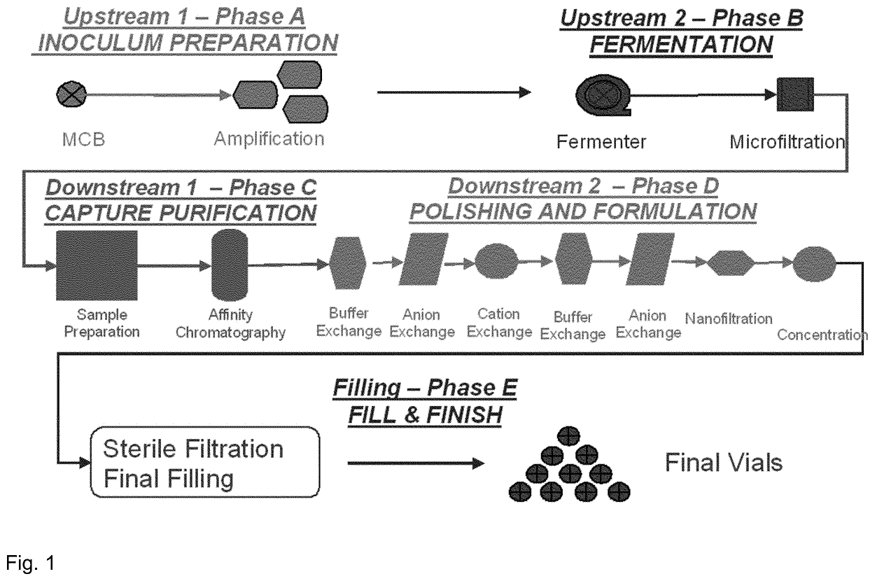

Both upstream phases are dedicated to cell culturing, whereas the downstream phases are for the recovery of L19-TNFα from the harvested medium by chromatographic purification. FIG. 1 shows a summarized process flow chart of the L19-TNFα production. The upstream and downstream processes are reported below.

### 1.1.1 Upstream Process

The Upstream process to produce L19-TNFα can be divided into two phases: Upstream 1 (phase A; inoculum preparation) and Upstream 2 (phase B; fermentation). Both phases can be divided into a number of steps:

| | |
|---|---|
| Step Nr. | Step description |

Upstream 1 (phase A)

| | |
|---|---|
| U1-10 | Seeding of L19-TNFα clone |
| U1-20 | Expansion of L19-TNFα clone from 1 T75 flask to 1 T150 flask |
| U1-30 | Expansion of L19-TNFα clone from 1 T150 flask to 3 T150 flasks |
| U1-40 | Expansion of L19-TNFα clone from 3 T150 flasks to 9 T150 flasks |
| U1-50 | Expansion of L19-TNFα clone from 9 T150 flasks to 4 roller bottles |
| U1-60 | Expansion of L19-TNFα clone from 4 roller bottles to 12 roller bottles |
| U1-70 | Expansion of L19-TNFα clone from 12 roller bottles to 36 roller bottles |
| U1-80 | Preparation of cells for seeding in the bioreactor |

Upstream 2 (phase B)

| | |
|---|---|
| U2-10 | Calibration of pH electrode bioreactor |
| U2-20 | Assembly and sterilization of the bioreactor |
| U2-30 | Transfer of culture medium into the bioreactor |
| U2-40 | Calibration pO₂ electrode of the bioreactor |
| U2-50 | Programming fermentation in the bioreactor |
| U2-60 | Inoculation of the bioreactor and start of fermentation |
| U2-70 | 1^{st} sterile sampling of the bioreactor (day 1) |
| U2-75 | Feed sterile assembly and scheduling (day 1) |
| U2-80 | 2^{nd} sterile sampling of the bioreactor (day 2) |
| U2-85 | Feed sterile assembly and scheduling (day 2) |
| U2-90 | 3^{rd} sterile sampling of the bioreactor (day 3) |
| U2-95 | Feed sterile assembly and scheduling (day 3) |
| U2-100 | 4^{th} sterile sampling of the bioreactor (day 4) |
| U2-105 | Feed sterile assembly and scheduling (day 4) |
| U2-110 | 5^{th} sterile sampling of the bioreactor (day 5) |
| U2-115 | Feed sterile assembly and scheduling (day 5) |
| U2-120 | 6^{th} sterile sampling of the bioreactor (day 6) |
| U2-130 | 1^{st} Harvest of the bioreactor and sterile sampling |
| U2-140 | Production gives “Clarified Bulk” worksheet to warehouse |
| U2-150 | 1^{st} sterile sampling of the bioreactor (day 1) |
| U2-155 | Feed sterile assembly and scheduling (day 1) |
| U2-160 | 2^{nd} sterile sampling of the bioreactor (day 2) |
| U2-165 | Feed sterile assembly and scheduling (day 2) |
| U2-170 | 3^{rd} sterile sampling of the bioreactor (day 3) |
| U2-175 | Feed sterile assembly and scheduling (day 3) |
| U2-180 | 4^{th} sterile sampling of the bioreactor (day 4) |
| U2-185 | Feed sterile assembly and scheduling (day 4) |
| U2-190 | 5^{th} sterile sampling of the bioreactor (day 5) |
| U2-195 | Feed sterile assembly and scheduling (day 5) |
| U2-200 | 6^{th} sterile sampling of the bioreactor (day 6) |
| U2-205 | Feed sterile assembly and scheduling (day 6) |
| U2-210 | 7^{th} sterile sampling of the bioreactor (day 7) |
| U2-220 | 2^{nd} Harvest of the bioreactor and sterile sampling |
| U2-230 | Production gives “Clarified Bulk” worksheet to warehouse |
| U2-240 | 1^{st} sterile sampling of the bioreactor (day 1) |
| U2-245 | Feed sterile assembly and scheduling (day 1) |
| U2-250 | 2^{nd} sterile sampling of the bioreactor (day 2) |
| U2-255 | Feed sterile assembly and scheduling (day 2) |
| U2-260 | 3^{rd} sterile sampling of the bioreactor (day 3) |
| U2-265 | Feed sterile assembly and scheduling (day 3) |
| U2-270 | 4^{th} sterile sampling of the bioreactor (day 4) |
| U2-275 | Feeds sterile assembly and scheduling (day 4) |
| U2-280 | 5^{th} sterile sampling of the bioreactor (day 5) |
| U2-285 | Feed sterile assembly and scheduling (day 5) |
| U2-290 | 6^{th} sterile sampling of the bioreactor (day 6) |
| U2-295 | Feed sterile assembly and scheduling (day 6) |
| U2-300 | 7^{th} sterile sampling of the bioreactor (day 7) |
| U2-310 | 3^{rd} Harvest of the bioreactor and sterile sampling |
| U2-320 | Production gives “Clarified Bulk” worksheet |
| U2-330 | 1^{st} sterile sampling of the bioreactor (day 1) |
| U2-335 | Feed sterile assembly and scheduling (day 1) |
| U2-340 | 2^{nd} sterile sampling of the bioreactor (day 2) |
| U2-345 | Feed sterile assembly and scheduling (day 2) |
| U2-350 | 3^{rd} sterile sampling of the bioreactor (day 3) |
| U2-355 | Feed sterile assembly and scheduling (day 3) |
| U2-360 | 4^{th} sterile sampling of the bioreactor (day 4) |
| U2-365 | Feed sterile assembly and scheduling (day 4) |
| U2-370 | 5^{th} sterile sampling of the bioreactor (day 5) |
| U2-375 | Feed sterile assembly and scheduling (day 5) |
| U2-380 | 6^{th} sterile sampling of the bioreactor (day 6) |
| U2-385 | Feed sterile assembly and scheduling (day 6) |
| U2-390 | 7^{th} sterile sampling of the bioreactor (day 7) - optional |
| U2-400 | 4^{th} Harvest of the bioreactor and sterile sampling |
| U2-410 | Production gives “Clarified Bulk” worksheet |
| U2-420 | 1^{st} sterile sampling of the bioreactor (day 1) - optional |
| U2-425 | Feed sterile assembly and scheduling (day 1) - optional |
| U2-430 | 2^{nd} sterile sampling of the bioreactor (day 2) - optional |
| U2-435 | Feed sterile assembly and scheduling (day 2) - optional |
| U2-440 | 3^{rd} sterile sampling of the bioreactor (day 3) - optional |
| U2-445 | Feed sterile assembly and scheduling (day 3) - optional |
| U2-450 | 4^{th} sterile sampling of the bioreactor (day 4) - optional |
| U2-455 | Feed sterile assembly and scheduling (day 4) - optional |
| U2-460 | 5^{th} sterile sampling of the bioreactor (day 5) - optional |
| U2-465 | Feed sterile assembly and scheduling (day 5) - optional |
| U2-470 | 6^{th} sterile sampling of the bioreactor (day 6) - optional |
| U2-475 | Feed sterile assembly and scheduling (day 6) - optional |
| U2-480 | 7^{th} sterile sampling of the bioreactor (day 7) - optional |
| U2-490 | 5^{th} Harvest of the bioreactor and sterile sampling - optional |
| U2-500 | Production gives “Clarified Bulk” worksheet to warehouse - optional |
| U2-510 | 1^{st} sterile sampling of the bioreactor (day 1) - optional |
| U2-515 | Feed sterile assembly and scheduling (day 1) - optional |
| U2-520 | 2^{nd} sterile sampling of the bioreactor (day 2) - optional |
| U2-525 | Feed sterile assembly and scheduling (day 2) - optional |
| U2-530 | 3^{rd} sterile sampling of the bioreactor (day 3) - optional |
| U2-535 | Feed sterile assembly and scheduling (day 3) - optional |
| U2-540 | 4^{th} sterile sampling of the bioreactor (day 4) - optional |
| U2-545 | Feed sterile assembly and scheduling (day 4) - optional |
| U2-550 | 5^{th} sterile sampling of the bioreactor (day 5) - optional |
| U2-555 | Feed sterile assembly and scheduling (day 5) - optional |
| U2-560 | 6^{th} sterile sampling of the bioreactor (day 6) - optional |
| U2-565 | Feed sterile assembly and scheduling (day 6) - optional |
| U2-570 | 7^{th} sterile sampling of the bioreactor (day 7) - optional |
| U2-580 | 6^{th} Harvest of the bioreactor and sterile sampling - optional |
| U2-590 | Production gives “Clarified Bulk” worksheet - optional |
| U2-600 | 1^{st} sterile sampling of the bioreactor (day 1) - optional |
| U2-605 | Feed sterile assembly and scheduling (day 1) - optional |
| U2-610 | 2^{nd} sterile sampling of the bioreactor (day 2) - optional |
| U2-615 | Feed sterile assembly and scheduling (day 2) - optional |
| U2-620 | 3^{rd} sterile sampling of the bioreactor (day 3) - optional |
| U2-625 | Feed sterile assembly and scheduling (day 3) - optional |
| U2-630 | 4^{th} sterile sampling of the bioreactor (day 4) - optional |
| U2-635 | Feed sterile assembly and scheduling (day 4) - optional |
| U2-640 | 5^{th} sterile sampling of the bioreactor (day 5) - optional |
| U2-645 | Feed sterile assembly and scheduling (day 5) - optional |
| U2-650 | 6^{th} sterile sampling of the bioreactor (day 6) - optional |
| U2-655 | Feed sterile assembly and scheduling (day 6) - optional |
| U2-660 | 7^{th} sterile sampling of the bioreactor (day 7) |
| U2-670 | Preparation of Post Production Cell Bank |
| U2-680 | End of fermentation (final harvest) and sampling |
| U2-690 | Production gives “Clarified Bulk” worksheet |
| U2-700 | Shutdown of the bioreactor |
| U2-710 | Final lot |

### Upstream 1 (Phase A)

EX-CELL CD CHO medium (provided by SAFC a unit of Sigma Aldrich; catalogue number 14361C-1000ML) with 6 mM L-Glutamine allows the cultivation or adaptation of recombinant CHO cells in a small scale (flasks). This medium was used in Upstream Phase 1, supplemented with 20 mM HEPES buffer when used in Roller Bottles.

### Upstream 2 (Phase B)

EX-CELL CD CHO medium with 6 mM L-Glutamine was also used for larger scale bioreactor fermentation in phase Upstream 2. Additionally, the feed supplement BalanCD CHO Feed 3 was added to the cell culture of L19-TNFα in phase Upstream 2. Furthermore, glucose 30% w/v solution was added to the cell culture to reach a concentration of 4 g/L glucose when the glucose concentration dropped below 2 g/L.

### Upstream 1 (Phase A): Inoculum Preparation for Fermentation

L19-TNFα cell culture began with the thawing of a master cell bank vial suitable for the production of L19-TNFα. The cell culture was first amplified in T75/T150 square flasks containing 15/30 ml of cell culture in standard conditions (37° C., 5% CO₂) and then expanded in 2 L roller bottles (37° C., 1.2 rpm) containing 250 ml of cell culture. EX-CELL CD CHO medium, supplemented with 6 mM Glutamine (6-XF), was used for square flask cell culturing. For cell culturing in roller bottles the same medium supplemented with 20 mM HEPES buffer (6-XRG) was used. The cell culture in square flasks and roller bottles was split every 3-4 days as detailed below. Amplifications were carried out at variable dilutions, to reach a viable cell concentration in the range of 100.000-150.000 cell/ml after dilution with fresh medium. To achieve a cell inoculum for bioreactor fermentation, the roller culture was expanded up to a total volume of 9 L (36 roller bottles) and to a cell concentration ≥500.000 cells/ml with a viability ≥60%.

Upstream 1 (phase A)

| | | Range of viable | | |
|---|---|---|---|---|
| | | cells | Step | Amplification |
| | | concentration | Duration | Time (day |
| Step Nr. | Step | (cell/ml) | (days) | nr.) |
| U1-10 | Seeding of L19-TNFα | — | 4 | 0 |
| U1-20 | Expansion of L19-TNFα from | 100.000-150.000 | 3 | 4 |
| | 1 T75 flask to 1 T150 flask | | | |
| U1-30 | Expansion of L19-TNFα from | 100.000-150.000 | 3 | 7 |
| | 1 T150 flask to 3 T150 flasks | | | |
| U1-40 | Expansion of L19-TNFα from | 100.000-150.000 | 4 | 10 |
| | 3 T150 flasks to 9 T150 flasks | | | |
| U1-50 | Expansion of L19-TNFα from | 100.000-150.000 | 4 | 14 |
| | 9 T150 flasks to 4 roller bottles | | | |
| U1-60 | Expansion of L19-TNFα from | 100.000-150.000 | 4 | 18 |
| | 4 roller bottles to 12 roller | | | |
| | bottles | | | |
| U1-70 | Expansion of L19-TNFα from | 100.000-150.000 | 3 | 22 |
| | 12 roller bottles to 36 roller | | | |
| | bottles | | | |
| U1-80 | Preparation of bioreactor | End of | 0 | 25 |
| | inoculum (pooling of cell culture | amplification, | | |
| | from roller bottles) | preparation of the | | |
| | | inoculums of the | | |
| | | bioreactor | | |

At the end of the amplification, the cell suspension was transferred into a sterile bag using a peristaltic pump. The sealed bag was then connected to the bioreactor using a steam-in-place stainless steel connector.

Upstream 2 (phase B): Fermentation

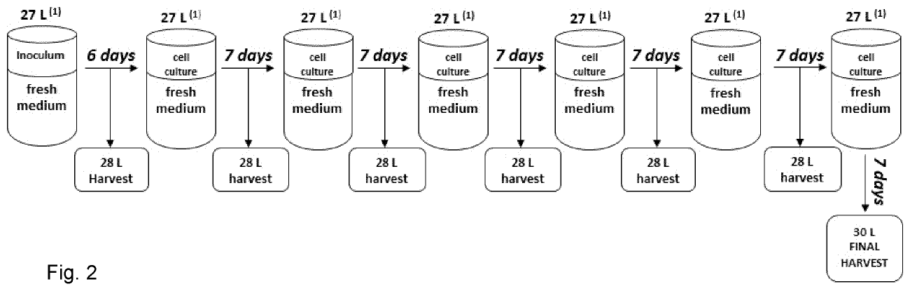

Fermentation took place in a Biostat C 30 bioreactor (B. Braun). The bioreactor was equipped with a gas mixer with 4 exits and a ring sparger aeration system. The pH was controlled through a CO₂ flow and calibrated by addition of a sodium carbonate solution. The stirrer was a single marine impeller. The temperature was controlled by a water jacket connected to a steam heat exchanger. The working volume was 30 L, the total volume of the vessel was 42 L. The semi-continuous fermentation process started when a portion of cell culture, obtained at the end of the Upstream 1 phase, was added to fresh medium in the bioreactor (inoculum). The fermentation of a single batch may take 27-48 days, according to the viability of the cell culture and the production requirements. Two twin bioreactors Biostat C 30 were used for fermentation in a semi-continuous approach in which each bioreactor was split every 6-7 days to harvest the cell culture. The harvested volume depended on the viable cell concentration determined before harvesting. To start each new fermentation cycle with a viable cell concentration of 100.000-200.000 cells/ml, enough cell culture was retained in the bioreactor to reach a final working volume of 27 L by adding fresh medium. As the process was performed in two twin Biostat C 30 reactors, both bioreactors were concurrently inoculated with portions of the cell culture obtained at the end of Upstream 1, running the semi-continuous fermentation simultaneously. The general fermentation scheme is outlined in FIG. 2. The operational steps of the Upstream 2 (phase B) process are summarized below.

Upstream 2 (phase B) (7 days fermentation)

| Fermentation | | | |
|---|---|---|---|
| time | Step | Step Duration | |
| (day) | Nr. | (days) | Action |
| 0 | U2-10 | 0 | Inoculum of the bioreactor from rollers' pool Upstream 1 (Phase A). |
| | to | | |
| | U2-60 | | |
| 1-6 | U2-70 | 6 | Fermentation in the bioreactor. |
| | to | | 1st harvest from the bioreactor and replacement with fresh medium. |
| | U2-140 | | |
| 7-13 | U2-150 | 7 | Fermentation in the bioreactor. |
| | to | | 2nd harvest from the bioreactor and replacement with fresh medium. |
| | U2-230 | | |
| 14-20 | U2-240 | 7 | Fermentation in the bioreactor. 3rd harvest from the bioreactor and |
| | to | | replacement with fresh medium. |
| | U2-320 | | |
| 21-27 | U2-330 | 7 | Fermentation in the bioreactor. 4th harvest from the bioreactor and |
| | to | | replacement with fresh medium. |
| | U2-410¹ | | |
| 28-34 | U2-420 | 7 | Fermentation in the bioreactor. 5th harvest from the bioreactor and |
| | to | | replacement with fresh medium. |
| | U2-500¹ | | |
| 35-41 | U2-510 | 7 | Fermentation in the bioreactor. 6th harvest from the bioreactor and |
| | to | | replacement with fresh medium. |
| | U2-590¹ | | |
| 42-48 | U2-600 | 7 | Fermentation in the bioreactor. 7th harvest from the bioreactor |
| | to | | |
| | U2-690¹ | | |
| 48 | U2-700 | 0 | Shutdown bioreactor |
| 48 | U2-710 | 0 | Lot Reconstitution |

¹Steps U2-410 to U2-655 are performed according to the viability of the cell culture and production requirements. Therefore, the fermentation in the bioreactors may be terminated after the 3^{rd}, 4^{th}, 5^{th} or 6^{th} harvesting.

### Inoculation of the Bioreactor

The bioreactor inoculum reaches a viable cell concentration in the range of 100.000-200.000 viable cells/ml at the beginning of the fermentation. The necessary dilution of medium and cell culture was calculated to reach a total working volume of 27 L. Before inoculation, the bioreactor was aseptically filled with the required quantity of culture medium (EX-CELL CHO Medium+6 mM Glutamine). The medium was transferred into the bioreactor vessel through the steam-in-place sterilized addition port device, passing a 0.22 μm filter with help of a peristaltic pump. The required quantity of cell culture, corresponding approximately to ¼ of total fermentation volume, was then inoculated into the bioreactor vessel to start the fermentation process. The aseptic addition of the inoculum was performed by gravity through the steam-in-place sterilized addition port device of the bioreactor.

### Fermentation Temperature

The temperature was kept at 37° C.±0.01° C. during the whole fermentation process.

### Culture pH

During fermentation the culture pH was kept stable at a pH of 7.20±0.02 by a flow of CO₂ for acid corrections (partial pressure: 1.25 atm) and by calibrated additions of a Na₂CO₃ 20% w/v solution for basic correction. Acid or base additions were regulated automatically by bioreactor management software through a gas mixer device (CO₂ addition) and a system peristaltic pump (Sodium Carbonate addition).

Dissolved Oxygen (pO₂)

The concentration of dissolved oxygen was maintained constant at 25%±2% air saturation value with a flow of air, nitrogen, and oxygen. The relative ratios of these three different gases were controlled by the fermentation management software through the gas mixer device of the bioreactor. Partial pressure of each: 0.95 atm for oxygen, nitrogen and air.

### Aeration and Stirring

The cell culture was aerated through a ring sparger diffusion system. The gas mixture comes out of the sparging device located on the base of the fermentation vessel through 1 mm holes. Bubbles are pushed down by the agitation system (single marine impeller) to increase the contact time of the gas mixture with the cell culture, thereby improving gas solubility. The impeller rotates at 88 rpm±5 during the first 24 hours of fermentation, then the rate is increased to 100 rpm±5 until the harvesting of the cell culture. The Airflow (gas mixture flow rate) set point is 1.0≤flow≤1.3 slpm±0.05 during the first 24 hours of every fermentation cycle, then the value is increased to 1.3≤flow≤1.5 slpm±0.05 until the harvesting of the cell culture.

### Semi Continuous Fermentation and Harvesting Volume

The fermentation process started after inoculation with the pool of roller bottles from Upstream 1 (phase A) with an initial working volume of 27 L. During each fermentation cycle, the cell culture was fed on day 1, 3 and 5 with the feed (5% total of the initial volume) as described in section Upstream 2 (phase B). The glucose concentration was regulated in order to reach 4 g/L of glucose concentration in the bioreactor. The fermentation was interrupted cyclically for intermediate harvesting of the cell culture after 6-7 days, harvesting about 30 L of cell culture, replaced with fresh medium to obtain a cell concentration in the range of 100.000-200.000 viable cells/ml at the beginning of each new fermentation cycle. At least 3 and up to a maximum of 7 splitting cycles were performed, depending on the viability of the cell culture and the production requirements. During the fermentation the cell culture was sampled aseptically daily. At the end of the 3^{rd}, 4^{th}, 5^{th}, 6^{th} or 7^{th} cycle, the fermentation process was terminated and the entire cell culture in the bioreactor was harvested. A complete fermentation process therefore lasted a total of 27 to 48 days, including equipment washing and sterilization. A total of seven harvests from each bioreactor can be obtained for a total Crude Harvest volume of about 215 L/bioreactor. An average of 8 g of crude product was obtained from a fermentation run.

### Microfiltration

Harvested material was clarified and sterilized using single-use cartridge filters. The first step of clarification was carried out with a 0.65 μm, 0.6 m² cartridge filter, while further clarification and sterilization was obtained through a combined 0.22 μm, 0.45 m² cartridge filter. The harvested material was loaded into the filters with a peristaltic pump at a flow rate of 1-2 L/min. Process yield was typically >90%. After filtration 0.5M EDTA solution pH 8.0 was added to the clarified harvest for a final EDTA concentration of 5 mM. The pH of the feedstock was adjusted to pH 7.5 to obtain the Clarified Bulk. The Clarified Bulk can be stored at 2-8° C. for up to two months until the next purification step.

### 1.1.2 Downstream Process

The downstream process for the purification of L19-TNFα included a series of chromatographic and (dia)filtration steps. The process is divided into Downstream 1 (phase C) and Downstream 2 (phase D), according to the following scheme:
a) Protein A affinity chromatography (Downstream 1)
b) Gel filtration (for buffer exchange) (Downstream 2)
c) Cation exchange chromatography (CIEX) (Downstream 2)
d) Gel filtration (for buffer exchange/formulation) (Downstream 2)
e) Anion exchange chromatography (AIEX) (Downstream 2)
f) Nanofiltration (for virus removal) (Downstream 2)
g) Diafiltration (for concentration) (Downstream 2)
h) Adding of Tween20 (for formulation completion) (Downstream 2).
   Downstream 1 (phase C)

The Downstream 1 process (phase C) consists of an affinity chromatography step using the rmp Protein A Sepharose Fast Flow, a recombinant multipoint ligand protein A resin. Multiple chromatographic runs were performed to process the entire amount of Clarified Bulk obtained at the end of the Upstream process. At the end of this step, the Semi-processed material was then purified in the Downstream 2 process or was otherwise stored at −80° C. for up to 60 days, after addition of 1.5% w/v glycerol. The purification scheme of Downstream 1 is shown below.

| Step | |
|---|---|
| Nr. | Step description |
| D1-10 | Preparation of the purification system |
| D1-20 | Purification of Clarified Bulk on rmpProtein A resin Sepharose Fast |
| | Flow, 1^{st} run |
| D1-30 | Purification of Clarified Bulk on rmpProtein A resin Sepharose Fast |
| | Flow, 2^{nd} run |
| D1-40 | Purification of Clarified Bulk on rmpProtein A resin Sepharose Fast |
| | Flow, 3^{rd} run |
| D1-50 | System shutdown |

### Buffer Preparation

Each buffer was prepared in a stainless-steel tank under mechanical agitation, and the pH was adjusted with 5 M NaOH or 5 M HCl. Buffers were then sterilized by filtration and transferred into sterile bags or bottles with the aid of a peristaltic pump. Buffers were stored at 2-8° C. and used within one month.

Affinity Chromatography on Protein a (Steps from D1-10 to D1-50)

The Protein A affinity chromatography was performed using the rmp Sepharose Fast Flow resin packed in a Vantage A2-180 column. The resin is a low leakage, non-mammalian-based affinity resin designed for high purity separation of monoclonal and polyclonal antibodies. The base matrix, Sepharose 4 Fast Flow, is a highly cross-linked, 4% agarose derivative, containing five antigen binding domains. The scFv (L19) moiety of L19-TNFα fusion protein binds to protein A immobilized on the resin. The volume of resin packed in the Vantage A2-180 column differed according to the Table below.

| Column | Min. volume | Max. volume |
|---|---|---|
| Vantage A2-180 | 800 ml | 2000 ml |

The volume of resin depended on the amount of sample to be purified; usually 1 ml of resin was used to purify up to 4.3 mg of L19-TNFα.

### Column Sanitization and Equilibration

Before sample loading, the column, resin and flow lines of the chromatographic system were sanitized in place with a solution of 0.1 M acetic acid/20% ethanol. The contact time was at least 60 minutes. After sanitization, the system was equilibrated with 2CV PBS PH 7.50 (NaH₂PO₄·2H₂O 50 mM; NaCl 150 mM, EDTA 5 mM, NaOH 50 mM), at a flow rate between 85 and 255 ml/min (corresponding to 20 and 60 cm/h).

### Sample Loading

After sanitization, the Clarified Bulk was loaded in fractions of approximately 80 L for each run, containing ca. 3800-4300 mg of L19-TNFα. Loading was at room temperature at a flow rate between 100 and 200 ml/min, corresponding to 24 and 48 cm/h, which allowed for a contact time of at least 8 minutes. The sample loading lasted approx. 14 hours.

### Sample Purification

Once the Clarified Bulk was loaded, the column was washed sequentially with the following buffers:
10 CV PBS/NaCl (NaH₂PO₄·2H₂O 50 mM; NaCl 650 mM, EDTA 5 mM, NaOH 53 mM, pH 7.50);
5 CV PBS pH 7.50 (NaH₂PO₄·2H₂O 50 mM; NaCl 150 mM, EDTA 5 mM, NaOH 50 mM).

The L19-TNFα fusion protein was then eluted from the column with TEA 10 mM pH 11.50 (TEA 10 mM, EDTA 5 mM, pH 11.50). Samples were eluted in a single fraction, ranging between 4 and 5 CV. The typical yield of this chromatographic step was higher than 90%. The chromatographic run was conducted by monitoring the A₂₈₀ₙₘ, the conductivity and the pH. The eluted sample (the Semi-processed material) was then dissolved in TEA 10 mM pH 11.50 (TEA 10 mM, EDTA 5 mM, pH 11.50) and incubated at room temperature for viral inactivation for 1 hour. The O.D. (A₂₈₀ₙₘ) at this stage ranged between 0.470 and 1.460.

The Semi-processed material was either processed in the next purification step, in the Downstream 2 process or was stored at −80° C., after addition of 1.5% w/v glycerol, for a maximum period of 60 days.

### Downstream 2 (Phase D)

The Downstream 2 process (phase D) consisted of a series of chromatographic and (dia)filtration steps, to purify and polish L19-TNFα. The Purified Bulk, obtained at the end of this process, represents the Drug Substance. The purification scheme of Downstream 2 is shown below.

| Step Nr. | Step description |
|---|---|
| D2-10 | Sephadex G25 system preparation |
| D2-20 | Buffer exchange of semi-processed material by Sephadex G25 |
| | (1^{st} run) |
| D2-30 | Buffer exchange of semi-processed material by Sephadex G25 |
| | (2^{nd} run) |
| D2-40 | Buffer exchange of semi-processed material by Sephadex G25 |
| | (3^{rd} run) |
| D2-50 | CHT type II 40 μm system preparation |
| D2-60 | Cation exchange of desalted semi-processed material by CHT |
| | type II 40 μm |
| D2-70 | Formulation of Purification Intermediate 1 by Sephadex G25 |
| D2-80 | Anion exchange of Purification Intermediate 2 by Sartobind Q |
| D2-90 | Nanofiltration of Purification Intermediate 3 by Viresolve |
| | ProModus |
| D2-100 | Concentration of Purification Intermediate 4 by Sartocon slice |
| | cassette system |
| D2-110 | Addition of 0.01% v/v Tween20 |

### Gel Filtration by Sephadex G25 (Buffer Exchange) (Steps D2-10 to D2-40)

### System Preparation

The semi-processed material (obtained from Downstream 1) underwent buffer exchange by gel filtration performed using the cross-linked dextran Sephadex G25 Fine resin packed in a Vantage A2-130 column. The volume of resin packed in the Vantage A2-130 column varied according to the Table below.

| Column | Min. volume | Max. volume |
|---|---|---|
| Vantage A2-130 | 3000 ml | 4500 ml |

The volume of resin used depended on the amount of protein to be processed; usually 1 ml of resin is used to purify up to 0.55 mg of L19-TNFα.

### Column Sanitization and Equilibration

Before sample loading, the column, resin and flow lines of the chromatographic system were sanitized in place with a solution of NaOH 0.2M. After sanitization, the system was equilibrated with 2 CV “Buffer phosphate 5 mM” solution (NaH₂PO₄·2H₂O 5 mM, pH 6.50), at a flow rate between 100 and 300 ml/min (corresponding to 45 and 135 cm/h).

### Sample Loading

After sanitization, the semi-processed material was loaded at a flow rate between 100 and 300 ml/min (corresponding to 45 and 135 cm/h). The semi-processed material was loaded in several fractions (generally 3-4 fractions), so that the load does not exceed the column's capacity (20% of column volume).

### Sample Elution

L19-TNFα was eluted (as desalted semi-processed material) with 0.6 CV “Buffer phosphate 5 mM” solution (NaH₂PO₄·2H₂O 5 mM, pH 6.50) at a flow rate between 100 and 300 ml/min (corresponding to 45 and 135 cm/h). The buffer exchange by gel filtration included a dilution of approx. 2-3 fold. The typical yield of this chromatographic step was 65-80%. The chromatographic run was conducted by monitoring A₂₈₀ₙₘ, conductivity and pH. The desalted semi-processed material can be stored at 2-8° C. for a maximum period of 72 hours.

Cation Exchange Chromatography (CIEX) (Steps from D2-50 to D2-60)

### System Preparation

The cation exchange chromatography (CIEX) was performed using a CHT ceramic hydroxyapatite type II (40 μm) resin, packed in a Vantage A2-60 column. Sets of five calcium doublets (C-sites) and pairs of hydroxyl-containing phosphate triplets (P-sites) were arranged in a repeating geometric pattern. The volume of resin packed in the column varied according to the Table below.

| Column | Min. volume | Max. volume |
|---|---|---|
| Vantage A2-60 | 500 ml | 1000 ml |

The volume of resin used depended on the amount of sample to be purified; usually 1 ml of resin is used to purify up to 2.7 mg of L19-TNFα.

### Column Sanitization and Equilibration

Before the sample loading, the column, resin and flow lines of the chromatographic system were sanitized with a solution of NaOH 1M. The contact time was at least 60 minutes. After sanitization, the system was equilibrated with 10 CV “Buffer A phosphate” solution (NaH₂PO₄·2H₂O 10 mM, pH 6.50), at a flow rate between 95 and 235 ml/min (corresponding to 200 and 500 cm/h). A Sartobind Q cartridge (0.250 m² filtration area) was installed in series before the CHT column. The Sartobind Q cartridge was separately activated with NaCl 1M, sanitized with NaOH 1M (60 minutes at least) and equilibrated with “Buffer phosphate 5 mM” solution (NaH₂PO₄·2H₂O 5 mM, pH 6.50).

### Sample Loading

After column sanitization, the desalted semi-processed material was loaded onto the system Sartobind Q-CHT column. The loading was performed at a flow rate between 95 and 235 ml/min (corresponding to 200 and 500 cm/h), which allowed for a contact time with the CHT resin of at least 2.5 minutes.

### Sample Elution

After loading, the CHT column was washed with 2 CV “Buffer A phosphate” solution (NaH₂PO₄·2H₂O 10 mM, pH 6.50). Then the L19-TNFα fusion protein was eluted from the column by a linear gradient, obtained by introducing progressively 40 CV “Buffer B phosphate” solution (NaH₂PO₄·2H₂O 10 mM, NaCl 1M, pH 6.50) from 0 to 100%, at a flow rate of 200 ml/min. The typical yield of this chromatographic step was around 70%. The chromatographic run was conducted by monitoring A₂₈₀ₙₘ, conductivity and pH. The purity of the Purification Intermediate 1, obtained at the end of the cation exchange chromatography, was higher than 98% for L19-TNFα total and higher than 95% for L19-TNFα homotrimer. The Purification Intermediate 1 can be stored at 2-8° C. for a maximum period of 48 hours.

### Gel Filtration by Sephadex G25 (Buffer Exchange/Formulation) (Step D2-70)

### System Preparation

The third operative step of Downstream 2 is the formulation of Purification Intermediate 1, performed through a buffer exchange by gel filtration chromatography. The Purification Intermediate 1 was processed to transfer the fusion protein L19-TNFα into its formulation buffer. The gel filtration was performed using the cross-linked dextran Sephadex G25 Fine resin, packed in a Vantage A2-130 column. The volume of resin which was packed in the Vantage A2-130 column varied according to the Table below.

| Column | Min. volume | Max. volume |
|---|---|---|
| Vantage A2-130 | 3000 ml | 4500 ml |

The volume of resin used depended on the amount of protein to be processed; usually 1 ml of resin is used to purify up to ca. 0.30 mg of L19-TNFα.

### Column Sanitization and Equilibration

Before sample loading, the column, resin and flow lines of the chromatographic system were sanitized in place with a solution of NaOH 0.2M. The contact time was at least 60 minutes. After sanitization, the system was equilibrated with 2 CV “Formulation buffer L19-TNFα without Tween20” (NaH₂PO₄·2H₂O 15 mM, Na₂HPO₄·2H₂O 10 mM, KCl 1.5 mM, Mannitol 75 mM, NaCl 30 mM, Glycerol 1% w/v, EDTA 5 mM, pH 8.00), at a flow rate between 100 and 300 ml/min (corresponding to 45 and 135 cm/h).

### Sample Loading

After sanitization, the Purification Intermediate 1 was loaded at a flow rate between 100 and 300 ml/min (corresponding to 45 and 135 cm/h). The Purification Intermediate 1 was loaded in several fractions (generally 5 f), as the load should not exceed the column's capacity (30% of column volume). Each fraction loading was followed by elution.

Sample elution

L19-TNFα was eluted as Purification Intermediate 2 with 0.6 CV “Formulation buffer L19-TNFα without Tween20” (NaH₂PO₄·2H₂O 15 mM, Na₂HPO₄·2H₂O 10 mM, KCl 1.5 mM, Mannitol 75 mM, NaCl 30 mM, Glycerol 1% w/v, EDTA 5 mM, pH 8.00) at a flow rate between 150 and 250 ml/min (corresponding to 70 and 113 cm/h). The buffer exchange by gel filtration included a dilution of around 1.5-fold. The typical yield of this chromatographic step was around 95%. The entire process generally lasted ca. 2 hours. The chromatographic run was conducted by monitoring A₂₈₀ₙₘ, conductivity and pH. The Purification Intermediate 2 was then directly processed in the next purification step of anion exchange chromatography.

### Anion Exchange Chromatography (AIEX) by Sartobind Q (Step D2-80)

The Purification Intermediate 2 was processed by anion exchange chromatography (AIEX), using a single use Sartobind Q capsule of 0.250 m² filtering area. Before sample loading, the capsule was activated flushing 200 ml NaCl 1M; then the capsule was sanitized by flushing NaOH 1M, for at least 60 minutes, and equilibrated by flushing “Formulation buffer L19-TNFα without Tween20” (NaH₂PO₄·2H₂O 15 mM, Na₂HPO₄·2H₂O 10 mM, KCl 1.5 mM, Mannitol 75 mM, NaCl 30 mM, Glycerol 1% w/v, EDTA 5 mM, pH 8.00) at a flow rate between 150 and 300 ml/min. The Purification Intermediate 2 was loaded at a flow rate between 150 and 250 ml/min. The L19-TNFα protein is positively charged (pH 8.0), therefore the Sartobind Q capsule was used in a flow through mode, collecting L19-TNFα directly into a container as Purification Intermediate 3. Possible contaminants negatively charged (such as residual DNA), were removed by binding to the Sartobind Q matrix. The typical yield of this chromatographic step was higher than 95%. The Purification Intermediate 3 can be stored at 2-8° C. for a maximum period of 48 hours, before the next purification step of nanofiltration.

### Nanofiltration (Virus Removal) (Step D2-90)

The Purification Intermediate 3 was processed for viral clearance by nanofiltration, using the Viresolve ProModus 1.1 or Viresolve ProModus 1.2 filters. The ProModus 1.1 or 1.2 can be used, with 0.017 and 0.070 m² filtering area, respectively. The membrane nominal cut-off is ca. 20 nm. Before use, the cartridge was wetted by flushing water for injection for 10 minutes at a constant pressure of 1.8 bar, it was then sanitized by flushing NaOH 0.5M for at least 60 minutes at the same pressure. After sanitization, the cartridge was equilibrated with “Formulation buffer L19-TNFα without Tween20” (NaH₂PO₄·2H₂O 15 mM, Na₂HPO₄·2H₂O 10 mM, KCl 1.5 mM, Mannitol 75 mM, NaCl 30 mM, Glycerol 1% w/v, EDTA 5 mM, pH 8.00) flushed at a constant pressure of 3 bar for 5 minutes. After equilibration, the Purification Intermediate 3 was loaded at a flow rate at ca. 300 ml/min (for Viresolve ProModus 1.2 filter). The L19-TNFα recovery after nanofiltration was usually higher than 99%. At the end of nanofiltration the Purification Intermediate 4 was obtained. If the optical density was lower than 0.530, the Purification Intermediate 4 was concentrated in the next Downstream 2 step. Otherwise, it was added directly with 0.01% v/v Tween20 to complete the Drug Substance formulation, obtaining the Purified Bulk.

### Diafiltration (for Concentration) (Step D2-100)

If the optical density was lower than 0.530, the Purification Intermediate 4 was concentrated by diafiltration, to adjust its optical density to a suitable range comprised between 0.530 and 0.580.

The diafiltration was performed using the Sartocon Slice cassette system. The system was sanitized by fluxing NaOH 1M for at least 60 minutes, before being rinsed with water for injection and equilibrated with “Formulation buffer L19-TNFα without Tween20” (NaH₂PO₄·2H₂O 15 mM, Na₂HPO₄·2H₂O 10 mM, KCl 1.5 mM, Mannitol 75 mM, NaCl 30 mM, Glycerol 1% w/v, EDTA 5 mM, pH 8.00), at a flow rate of ca. 50 ml/min. When the equilibration was complete, the Purification Intermediate 4 was loaded into the system. The Purification Intermediate 4 was continuously flushed through the Sartocon cassette. L19-TNFα was retained and returned to the starting bottle “Retentate”, whereas the formulation buffer, which passes the membrane, was discarded in the bottle “Permeate”. Since the buffer was not replaced, the L19-TNFα concentration progressively increased. During the concentration, the Purification Intermediate 4 volume decreased approx. 2-4 fold, according to the starting optical density. The process was controlled by monitoring the optical density value. When it reached the established range 0.530-0.580, the concentration process was stopped. The yield of this operative step was generally higher than 99%. The obtained Purification Intermediate 5 can then be directly added with 0.01% v/v Tween20 to complete the Drug Substance formulation.

Addition of 0.01% v/v Tween20 (step D2-110)

The Drug Substance formulation is obtained by adding 0.01% v/v Tween20. A stock solution of Tween20 10% v/v was added in a suitable quantity, to reach the final content 0.01% v/v Tween20. Tween20 was added to the Purification Intermediate 4 at the end of the nanofiltration, if the optical density was already in the range 0.530-0.580, otherwise it was added to the Purification Intermediate 5, after the completion of the concentration process. The addition of 0.01% v/v Tween20 completed the Downstream 2 process. The Purified Bulk (Drug Substance) can be stored at 2-8° C. for a maximum period of 7 days or at −80° C. for up to 1 month.

### Example 2—Quality Control of GMP Lots

Purity of the good manufacturing process (GMP) lots prepared according to Example 1 were characterized using several quality control analyses, including ELISA, SDS-PAGE, Size Exclusion Chromatography, A₂₈₀ measurements, productivity, bioactivity and immunoreactivity assays, osmolality, viral testing, visual appearance etc. The protocol used for 2D-SDS-PAGE analysis, is reported below.

### 2.1 2D-SDS-PAGE

Each sample was analyzed in a bidimensional electrophoresis assay using a precast system (ZOOM® IPGRunner™). The assay was performed by adopting two ranges of pH for iso-electric focusing:

$$1 ) pH 3. - 10. \\ 2 ) pH 6. - 10. .$$

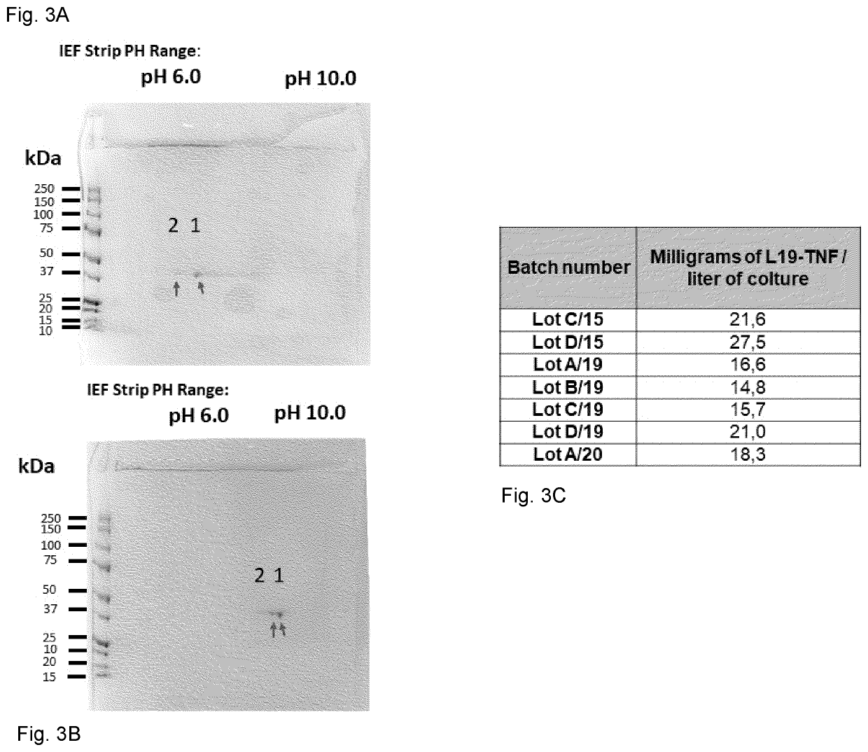

In (1), the pH range is wide enough to underline eventual contaminants while in (2) the pH range separation is smaller but still includes the isoelectric point of the sample (pH=7.7). Staining was performed with Blue Coomassie R-250. The results are reported in FIG. 3.

### Example 3—Mass Spectrometry Analysis of GMP Lots

### 3.1 Direct Infusion HR-MS for Intact Mass Analysis

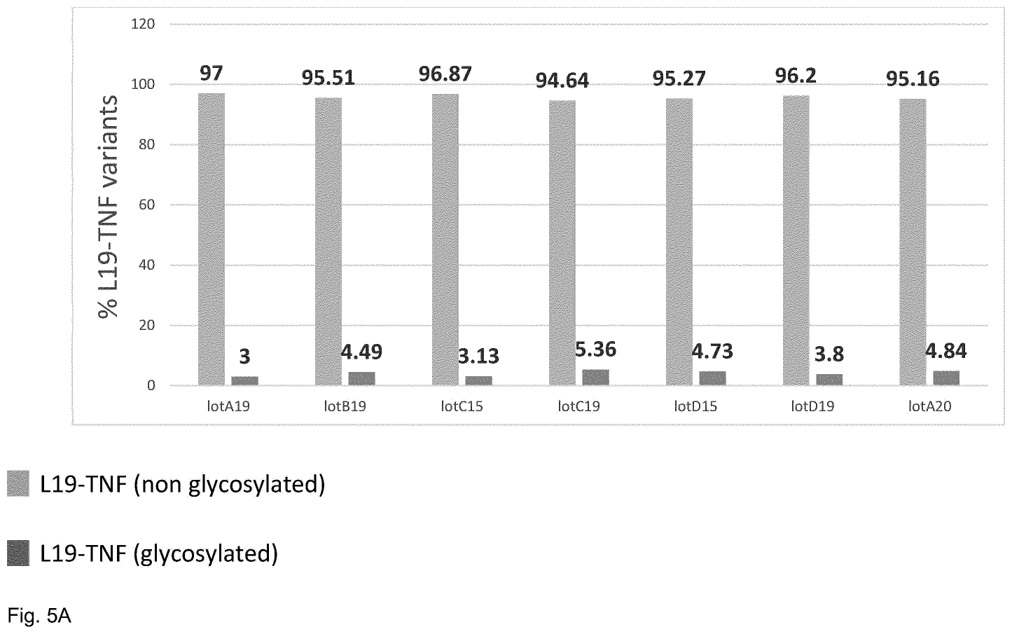

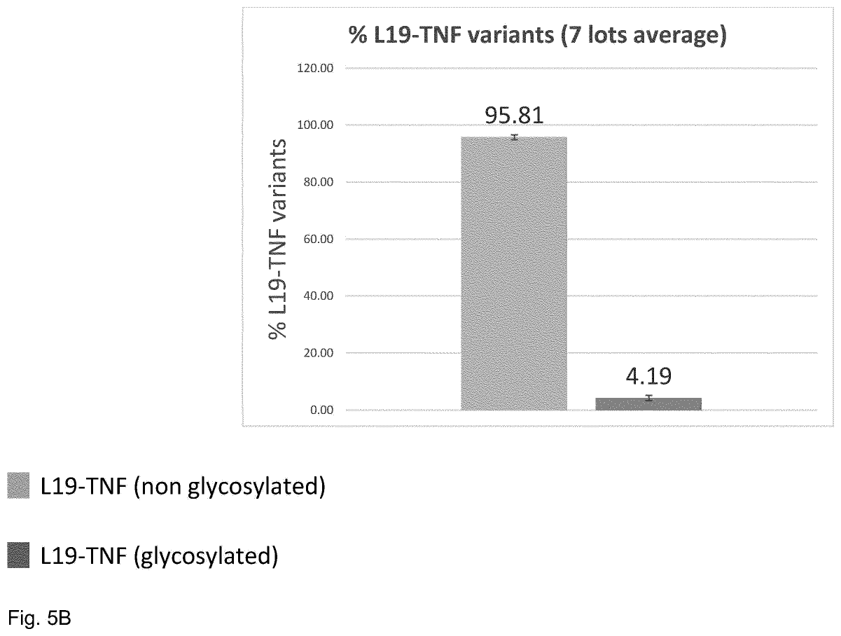

100 μg of L19-TNFα samples were desalted using C18-purification before analysis (Micro Spin Columns, Harvard Apparatus). Direct infusion was performed on an Orbitrap Q-Exactive coupled to an Ion Max ESI source. The following parameters were used: syringe flow rate 4 μL/min, capillary voltage 3.0 kV, in source induced dissociation 40 eV, sheath gas 4 units, capillary temperature 300° C., S-lens RF level 90, resolution 17500 (FWHM at 200 m/z), AGC target 5×10⁴, microscan 10, mass range 500-3000 m/z, and maximum injection time 200 ms. Raw spectra were then analyzed to quantify relative amounts of glycosylated and non-glycosylated L19-TNFα of the total L19-TNFα in the preparation. Quantification was achieved by summing the intensities of the peaks corresponding to glycosylated or non-glycosylated L19-TNFα, respectively, and the percentage was calculated using the equation below.

$$\frac{Intensity of x}{\sum Intensities} \times 100$$

Where x is the sum of the intensities corresponding to non-glycosylated or glycosylated L19-TNFα, respectively. The results of this analysis are shown in FIGS. 4 and 5.

### 3.2 MS/MS Analysis for Glycopeptide Analysis

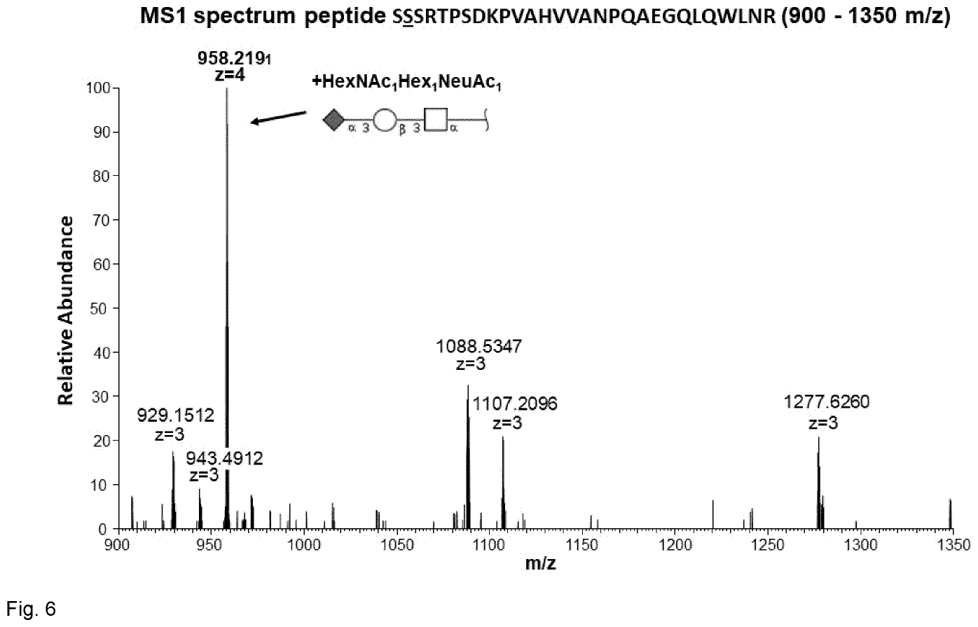

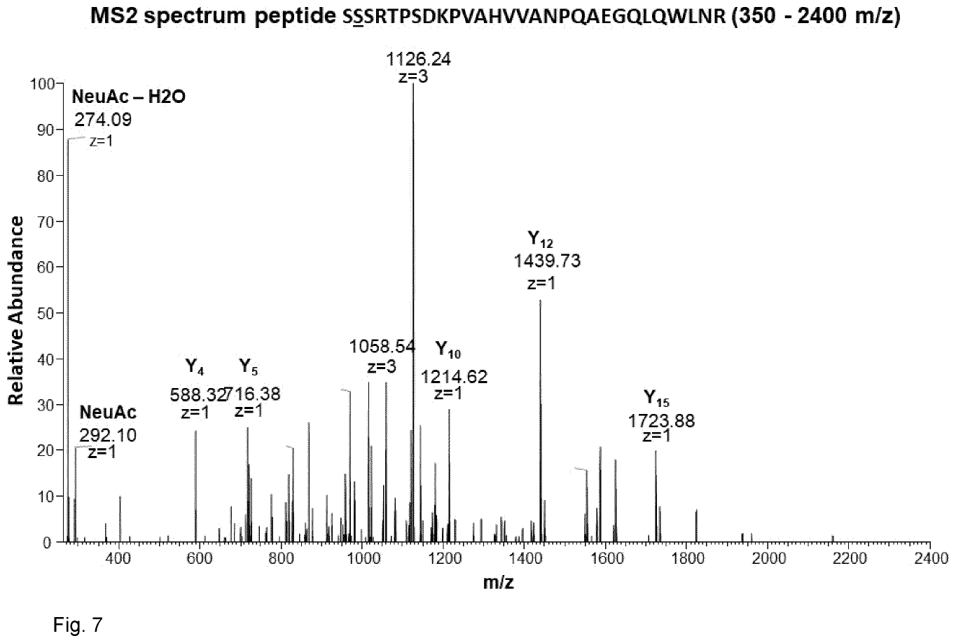

7.5 μg of L19-TNFα were resuspended in urea 1M dissolved in NH₄HCO₃ solution at a final pH=8. Protein was reduced with TCEP for 15 min at RT followed by 45 min at 65° C. and alkylated with Iodoacetamide (IAA) for 1 hour in the dark. L19-TNFα was then digested by trypsin (enzyme-protein ratio 1:60) at 37° C. overnight. After digestion, the sample was acidified with 10% formic acid and then subjected to C18 purification and desalting (Macro Spin Columns, Harvard Apparatus). 500 ng of the resulting peptides were then subjected to HPLC-MS/MS analysis. All samples were analysed on an Orbitrap Q-Exactive mass spectrometer coupled to an EASY nanoLC 1000 system via a Nano Flex ion source. Chromatographic separation was carried out on an Acclaim PepMap RSLC column (50 μm×15 cm, particle size 2 μm, pore size, 100 Å), using 40 min linear gradient with 5-35% solvent B (0.1% formic acid in acetonitrile) at a flow rate of 300 nL/min. Ionization was carried out in positive ion mode, with 2 kV of spray voltage, 250° C. of capillary temperature, 60 S-lens RF level. The mass spectrometer was working in a data-dependent mode. MS1 scan range was set from 350 to 1650 m/z, the 10 most abundant peptides were subjected to HCD fragmentation with NCE of 25. A dynamic exclusion was set at 10 seconds. Raw files were processed with Proteome Discoverer 1.4. Database searches were performed using Sequest as search engine using a FASTA file containing our protein of interest, the Mus musculus reference proteome and additional contaminants (human keratin isoforms, bovine serum albumin and ProteinA from Staphylococcus Aureus). Carbamidomethylation of cysteines was set as a fixed modification while oxidation of methionine and the O-glycosylation (HexNAc₁Hex₁NeuAc₁) were set as variable modifications. Trypsin was set as cleavage specificity, allowing a maximum of 2 missed cleavages. Data filtering was performed using percolator, resulting in 1% false discovery rate (FDR). The results of this analysis are reported in FIGS. 6 and 7.

Sequence Listing
Amino acid sequence of L19 CDRs

| | |
|---|---|
| L19 CDR1 VH-SFSMS | (SEQ ID NO: 1) |
| L19 CDR2 VH-SISGSSGTTYYADSVKG | (SEQ ID NO: 2) |
| L19 CDR3 VH-PFPYFDY | (SEQ ID NO: 3) |
| L19 CDR1 VL-RASQSVSSSFLA | (SEQ ID NO: 4) |
| L19 CDR2 VL-YASSRAT | (SEQ ID NO: 5) |
| L19 CDR3 VL-QQTGRIPPT | (SEQ ID NO: 6) |

Amino acid sequence of the L19 VH domain (SEQ ID NO: 7)
EVQLLESGGGLVQPGGSLRLSCAASGFTFSSFSMSWVRQAPGKGLEWVSSISGSSGTTYYAD
SVKGRFTISRDNSKNTLYLQMNSLRAEDTAVYYCAKPFPYFDYWGQGTLVTVSS
Amino acid sequence of the linker between VH and VL (SEQ ID NO: 8)
GDGSSGGSGGAS
Amino acid sequence of the L19 VL domain (SEQ ID NO: 9)
EIVLTQSPGTLSLSPGERATLSCRASQSVSSSFLAWYQQKPGQAPRLLIYYASSRATGIPDRFS
GSGSGTDFTLTISRLEPEDFAVYYCQQTGRIPPTFGQGTKVEIK
Amino acid sequence of the L19 scFv (SEQ ID NO: 10)
EVQLLESGGGLVQPGGSLRLSCAASGFTFSSFSMSWVRQAPGKGLEWVSSISGSSGTTYYAD
SVKGRFTISRDNSKNTLYLQMNSLRAEDTAVYYCAKPFPYFDYWGQGTLVTVSSGDGSSGGS
GGASEIVLTQSPGTLSLSPGERATLSCRASQSVSSSFLAWYQQKPGQAPRLLIYYASSRATGIP
DRFSGSGSGTDFTLTISRLEPEDFAVYYCQQTGRIPPTFGQGTKVEIK
Amino acid sequence of the soluble form of the extracellular domain
of human TNFα (huTNFα) (SEQ ID NO: 11).
VRSSSRTPSDKPVAHVVANPQAEGQLQWLNRRANALLANGVELRDNQLVVPSEGLYLIYSQVL
FKGQGCPSTHVLLTHTISRIAVSYQTKVNLLSAIKSPCQRETPEGAEAKPWYEPIYLGGVFQLEK
GDRLSAEINRPDYLDFAESGQVYFGIIAL
Amino acid sequence of the linker between scFv and TNFα (SEQ ID
NO: 12)
EFSSSSGSSSSGSSSSG
Amino acid sequence of the L19-huTNFα conjugate (SEQ ID NO: 13)
Serine 257 is shown in bold, italics and underlined
EVQLLESGGGLVQPGGSLRLSCAASGFTFSSFSMSWVRQAPGKGLEWVSSISGSSGTTYYAD
SVKGRFTISRDNSKNTLYLQMNSLRAEDTAVYYCAKPFPYFDYWGQGTLVTVSSGDGSSGGS
GGASEIVLTQSPGTLSLSPGERATLSCRASQSVSSSFLAWYQQKPGQAPRLLIYYASSRATGIP
DRFSGSGSGTDFTLTISRLEPEDFAVYYCQQTGRIPPTFGQGTKVEIKEFSSSSGSSSSGSSSS
GVRSSSRTPSDKPVAHVVANPQAEGQLQWLNRRANALLANGVELRDNQLVVPSEGLYLIYSQ
VLFKGQGCPSTHVLLTHTISRIAVSYQTKVNLLSAIKSPCQRETPEGAEAKPWYEPIYLGGVFQL
EKGDRLSAEINRPDYLDFAESGQVYFGIIAL
Amino acid sequence of the L19-IL2 conjugate (SEQ ID NO: 14)
Threonine 256 is shown in bold, italics and underlined
EVQLLESGGGLVQPGGSLRLSCAASGFTFSSFSMSWVRQAPGKGLEWVSSISGSSGTTYYAD
SVKGRFTISRDNSKNTLYLQMNSLRAEDTAVYYCAKPFPYFDYWGQGTLVTVSSGDGSSGGS
GGASEIVLTQSPGTLSLSPGERATLSCRASQSVSSSFLAWYQQKPGQAPRLLIYYASSRATGIP
DRFSGSGSGTDFTLTISRLEPEDFAVYYCQQTGRIPPTFGQGTKVEIKEFSSSSGSSSSGSSSS
GAPTSSSTKKTQLQLEHLLLDLQMILNGINNYKNPKLTRMLTFKFYMPKKATELKHLQCLEEELK
PLEEVLNLAQSKNFHLRPRDLISNINVIVLELKGSETTFMCEYADETATIVEFLNRWITFCQSIIST
LT

1. A pharmaceutical preparation comprising:
(i) non-glycosylated L19-TNFα; and
(ii) glycosylated L19-TNFα;
wherein the L19-TNFα comprises or consists of the sequence set forth in SEQ ID NO: 13, and wherein the percentage of glycosylated L19-TNFα is ≤6% of the total L19-TNFα in the preparation.

2. The pharmaceutical preparation of claim 1, wherein the glycosylated L19-TNFα comprises an O-linked glycosylation on the serine at position 257 of SEQ ID NO: 13.

3. The pharmaceutical preparation of claim 2, wherein the O-linked glycosylation consists of HexNAc₁Hex₁NeuAc₁.

4. The pharmaceutical preparation according to any one of claims 1 to 3, wherein the percentage of glycosylated L19-TNFα is determined by mass spectrometry analysis, preferably intact mass spectrometry analysis.

5. The pharmaceutical preparation according to any one of claims 1 to 4, further comprising a pharmaceutically acceptable excipient, carrier, buffer, stabiliser.

6. The pharmaceutical preparation according to any one of claims 1 to 5, for use in a method of treating cancer in a patient.

7. The pharmaceutical preparation for use according to claim 6, wherein the cancer is a melanoma glioma or skin cancer, such as a malignant melanoma or non-melanoma skin cancer.

8. The pharmaceutical preparation for use according to claim 6 or 7, wherein the method comprises administering a second anti-cancer therapeutic to the patient.


SEQUENCE LISTING (15 sequences)

SEQ ID NO: 1 - protein, 5 aa, synthetic construct
  REGION 1..5: note = L19 CDR1 VH
SFSMS

SEQ ID NO: 2 - protein, 17 aa, synthetic construct
  REGION 1..17: note = L19 CDR2 VH
SISGSSGTTYYADSVKG

SEQ ID NO: 3 - protein, 7 aa, synthetic construct
  REGION 1..7: note = L19 CDR3 VH
PFPYFDY

SEQ ID NO: 4 - protein, 12 aa, synthetic construct
  REGION 1..12: note = L19 CDR1 VL
RASQSVSSSFLA

SEQ ID NO: 5 - protein, 7 aa, synthetic construct
  REGION 1..7: note = L19 CDR2 VL
YASSRAT

SEQ ID NO: 6 - protein, 9 aa, synthetic construct
  REGION 1..9: note = L19 CDR3 VL
QQTGRIPPT

SEQ ID NO: 7 - protein, 116 aa, synthetic construct [sequence omitted]

SEQ ID NO: 8 - protein, 12 aa, synthetic construct
  REGION 1..12: note = Linker between VH and VL
GDGSSGGSGGAS

SEQ ID NO: 9 - protein, 108 aa, synthetic construct [sequence omitted]

SEQ ID NO: 10 - protein, 236 aa, synthetic construct
  REGION 1..236: note = L19 scFv
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 11 - protein, 157 aa, synthetic construct [sequence omitted]

SEQ ID NO: 12 - protein, 17 aa, synthetic construct
  REGION 1..17: note = Linker between scFv and TNFalpha
EFSSSSGSSSSGSSSSG

SEQ ID NO: 13 - protein, 410 aa, synthetic construct
  REGION 1..410: note = L19-huTNFalpha conjugate
  CARBOHYD 257: note = S is linked to O-linked glycan structure HexNAc1Hex1NeuAc1
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 14 - protein, 386 aa, synthetic construct
  REGION 1..386: note = L19-IL2 conjugate
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 15 - protein, 29 aa, synthetic construct
  REGION 1..29: note = Glycosylated TNF alfa peptide
  CARBOHYD 2: note = S is linked to O-linked glycan structure HexNAc1Hex1NeuAc1
SSSRTPSDKPVAHVVANPQAEGQLQWLNR